(19) United States
(12) Patent Application Publication
(10) Pub. No.: US 2010/0267680 A1
(43) Pub. Date: Oct. 21, 2010

(54) USE OF AT LEAST ONE OXIME DERIVATIVE OF CHOLEST-4-EN-3-ONE AS ANTIOXIDANTS

(76) Inventors: Rebecca Pruss, Cassis (FR); Cyrille Drouot, Draguignan (FR)

Correspondence Address: BROWDY AND NEIMARK, P.L.L.C.;624 NINTH STREET, NW, SUITE 300, WASHINGTON, DC, 20001-5303, US

(21) Appl. No.: 12/670,459

(22) PCT Filed: Jul. 24, 2008
(86) PCT No.: PCT/FR08/01101

§ 371 Date: Jun. 25, 2010

(30) Foreign Application Priority Data
Jul. 25, 2007 (FR) 0705426

(51) Int. Cl. A61K 8/63; A61K 47/28; A61Q 19/08; A23L 1/30; A23L 3/3463

(52) U.S. Cl. 514/169; 514/788; 426/544; 426/2

(57) ABSTRACT

The invention relates to the use of at least one oxime derivative of cholest-4-en-3-one as antioxidants in the cosmetics and food fields, and as antioxidant preservatives that can be used, in particular, in cosmetic, food and pharmaceutical products.

The present invention relates to the use of at least one oxime derivative of cholest-4-en-3-one for its antioxidant property. More particularly, the present invention relates to the use of at least one oxime derivative of cholest-4-en-3-one as antioxidants in the cosmetics and food fields, and as an antioxidant preservative which can be used in particular in cosmetic, food, and pharmaceutical products.

Oxidative stress is one of the biological consequences of the use of oxygen by the organism. It leads to the formation of free radicals in the cells. Free radicals, if not controlled, can rapidly react with molecules surrounding them, giving rise to toxic compounds which can interfere with normal physiological processes. These substances can lead to cell damage if the antiradical defences are insufficient. More and more studies show that reactive oxygen species play an important role in multiple biological processes and in particular in the development of multiple human pathologies, and in ageing.

The cumulative effects of these reactions can overwhelm the normal cell repair mechanisms.

The role of cell oxidation in ageing and particularly cutaneous ageing, whether intrinsic or extrinsic, in particular light-induced, is known. Cutaneous ageing manifests itself by different clinical signs in particular the appearance of fine lines and deep wrinkles, increasing with age. Moreover, the appearance of the skin or the scalp deteriorates. The skin tone is generally altered and there may be diffuse irritations and sometimes telangiectasias on certain areas of the skin.

Another clinical sign of ageing is the dry and rough appearance of the skin which is essentially due to greater desquamation. Finally, a loss of firmness and tonicity of the skin is noted which, as with wrinkles and fine lines, is at least partly explained by a dermal and epidermal atrophy as well as a flattening of the formation. It is therefore noted that the clinical signs of cutaneous ageing result essentially from a dysfunction of the main biological mechanisms involved in the skin.

Preventing or treating cutaneous ageing, whether intrinsic or extrinsic, and the clinical signs described above, comes down to maintaining or improving the appearance of the skin or scalp.

Different antioxidants capable of preventing or treating cutaneous ageing are described in the state of the art.

Antioxidants are substances which neutralize the free radicals or their actions. Thus, they help to protect cells against the damage caused by free radicals.

The natural antioxidant molecules include for example vitamins (A, E and C in particular), carotenoids (such as beta-carotene), polyphenols, and trace elements (such as selenium, copper and zinc).

The beneficial effect of an exogenous supply of antioxidant to limit oxidative stress and reinforce the antioxidant defence, by ingestion, is known. Recent scientific data have shown that, in certain animal species, the administration of antioxidants effectively halts the ageing process and increases the animal's longevity.

It is thus sometimes beneficial, in order to allow the organism to function normally, to ingest components possessing an antioxidant action in a sufficient quantity.

Antioxidants also have a beneficial effect when applied to the skin, they are in fact used in cosmetics.

The use of antioxidants as preservatives, in various types of products sensitive to oxidation, is also known.

However, the compounds used as antioxidants are sometimes inappropriate or have an insufficient action. It is known for example that tocopherol which is a reference product in this field is sensitive to light and requires particular preservation means.

There is still therefore a real need for antioxidant compounds, and it would be useful to have new antioxidants having a powerful antioxidant activity which would have a beneficial effect in the cosmetics field, in the food field, and also an effect on the preservation of products.

The present invention is a response to this demand for powerful antioxidant compounds since it involves the use of derivatives of cholest-4-en-3-one, which are powerful antioxidants, as antioxidants.

In fact the inventors have now shown the powerful antioxidant role of at least one oxime derivative of cholest-4-en-3-one, and in particular cholest-4-en-3-one oxime vis-à-vis the peroxidation of lipids and also vis-à-vis the substances capable of undergoing heat- or light-induced oxidation reactions (such as proteins, sugars, pigments, vitamins, polymers).

This is why a subject of the present invention is the use of at least one oxime derivative of cholest-4-en-3-one as an antioxidant.

According to the invention the term “antioxidant” refers to the ability of a compound to reduce the damage caused by free radicals:
in the organism, as an active ingredient in the cosmetics field and in the food field, and
in any type of product requiring it as a preservative in order to be better preserved.

Any use of the compounds as active ingredients for a therapeutic application is excluded.

In addition to their excellent antioxidant properties, these compounds have the following advantages:
their synthesis is inexpensive;
they exhibit no proven toxicity during oral administration over 9 months in high doses in animals, i.e. they can be used in food as well as in pharmaceutical, dermatological or cosmetic products without this presenting any health or toxicological problem;
because they are powerful antioxidants, the necessary dose is very low;
these compounds do not absorb in the UV/visible region, they do not therefore interfere with conventional sun products which absorb UV (ultraviolet) rays and present no risk of chemical instability in this wavelength range;
these compounds are presented in the form of crystalline powder and can be stored very well at ambient temperature, with no degradation for at least 24 months;
they possess very good solubility in fats;
they are colourless, tasteless and odourless, which is an advantage for use in the food and cosmetics fields in particular;
they are bioavailable, which makes them compounds which can be expected to have a systemic activity by oral route;
their membrane destination (BORDET et al., J. Pharmacol. Exp. Ther., 322: 709-720 (2007)) after ingestion and assimilation makes them excellent candidates for the protection of membrane components particularly lipids against peroxidation.

The antioxidant properties of the compounds of the invention make them suitable for use in the cosmetics field.

Thus, a first aspect of the invention is the use of at least one oxime derivative of cholest-4-en-3-one to protect the skin.

The skin is in particular the site of attack by extrinsic and intrinsic toxic factors. The extrinsic factors include for example ultraviolet radiation, wind, low humidity, abrasives and strong surfactants. The intrinsic factors include chronological ageing and biochemical changes in the skin.

A cause-effect relationship exists between repeated exposure to UV and premature ageing of the skin. Excessive exposure to the sun contributes to a premature reduction in the quality and quantity of elastin and collagen in the skin, and to hypertrophy of the epidermis. These changes are manifested by typical signs of ageing, such as wrinkles, a loss of elasticity, a dryness of the skin and a greater frequency of spots, and benign or malignant neoplasias.

The compounds of the present invention are capable of providing effective protection against the factors which cause the appearance of wrinkles and other histological changes associated with ageing of the skin.

It is therefore also one of the subjects of the invention to use the antioxidant properties of the compounds according to the invention on the symptoms of ageing due to UV, i.e. on the damage to the skin which appears as the result of repeated exposure to the sun in order to prevent, remove and treat wrinkles, fine lines of the skin, and/or combat cutaneous and/or subcutaneous relaxation; and/or improve the texture of the skin and revive the lustre of the skin; and/or reduce the size of the pores of the skin.

The useful properties of the compounds of the invention, their zero absorption in the UVA and very low absorption in the UVB spectrum, also justify their use in a sun-protection cream, with no risk of interfering with the action of the components especially chosen for their UV absorption. The compounds of the invention are capable of trapping the form of oxygen activated by solar radiation. This activated form of oxygen, called singlet oxygen, is the reactive entity at the origin of cell disorders.

Another aspect of the invention consists of the use of the compounds of the invention in cleansing and/or make-up removal products, as well as in products for protection of the skin and/or hair against the side effects of UV.

The antioxidant properties of at least one oxime derivative of cholest-4-en-3-one also make them suitable for use in the food field in the form of a food supplement. It is therefore within the scope of the invention to use the compounds of the invention as antioxidants in the food field.

By antioxidant in the food field is meant in the present invention, a compound which, in the pure form or mixed with various supports and/or other permitted food additives, can be presented in powder form, in the form of gelatin capsules, tablets or other solid form which can optionally comprise a lipid, aqueous phase or be in oral solution or suspension.

Advantageously, the compound can be consumed alone, between meals, or during meals.

In advantageous manner, it can be consumed during meals, as a food supplement, combined with other foods. It is preferably incorporated in or sprinkled on a food. In practice the foods can be simple or mixed foods, and can be presented in all the usual forms known for human consumption. By food is meant within the meaning of the present invention, any food which can be ingested alone or accompanied, raw or cooked, prepared or not prepared, in any way whatever, such as for example meats and meat-based products, sea and freshwater products, milk and dairy products, including infant's milk, eggs and egg products, fruit and vegetables, cereals and cereal-based products, starchy products such as dough and rice, oils, vinegars and condiments, sauces and edible fats, sweetened products, jams, jellies, compotes, spreads, confectionary, preserves and semi-preserves, soups, coffee, tea, beverages, pastry, cocoa, chocolate, ices, meal replacements, ready-made and freshly prepared, quick-frozen or sterilized meals, bread and breadmaking products.

Thus, the compound can accompany any food without interfering with the taste and does not constitute a constraint for the consumer. Taking it can be seen as part of the food preparation process. It is not like taking medicaments or eating substitute meals. The compounds according to the invention can be frozen or, by contrast, heated without losing their properties.

The invention also relates to the use of the compounds according to the invention as antioxidant preservatives in different products, in particular cosmetic, food, and pharmaceutical products.

By preservative is meant a compound which keeps a product from any physico-chemical alteration.

It is known that fats and certain active substances used in cosmetic, dermatological, pharmaceutical, or detergent compositions in particular, have a tendency to oxidize, even at ambient temperature, and that this oxidation causes them to acquire new, in particular olfactory, properties which are undesirable. It is known for example that certain soaps develop rancid, spicy and fruity odours after only a few weeks of storage in the air. These unpleasant odours can be prevented or at least avoided for a much longer storage period if one of the compounds of the invention is added to them. Similar effects have been observed with shampoos or also shower or bath gels, cosmetic creams and lotions, cosmetic or skin or hair cleansing products containing substances which can oxidize in the air and/or in the light. It is within the scope of the invention to use the compounds of the invention as antioxidant agents for preserving cosmetic, dermatological, pharmaceutical, detergent and fragrance products.

Similarly, food products degrade under the action of oxidation in air, which causes changes in texture, colour and taste, and can make a food unfit for consumption.

The applicant has discovered that the compounds of the invention make it possible to ensure better preservation of the cosmetic or dermatological compositions comprising a oil phase, avoiding the rancidity of unsaturated lipids contained therein, and that they could also make it possible to avoid the oxidative degradation of active compounds contained in these compositions, such as vitamin A or the carotenoids.

It is therefore within the scope of this invention to use the compounds of the invention as preservatives, particularly for the preservation of the organoleptic and nutritional properties of foods and drinks, in particular fruit juices.

As the compounds according to the invention have high antioxidant capacities, they can be used as antioxidant preservatives of any lipid-based preparation including food, cosmetic, dermatological, fragrance, detergent products or pharmaceutical products.

A subject of the present invention is therefore the use of compounds according to the invention as preservatives, in particular in cosmetic or dermatological products, and food products.

This is why a subject of the present invention is the use of at least one oxime derivative of cholest-4-en-3-one or one of its addition salts with acceptable acids, or one of its esters or one of the addition salts of said esters with acceptable acids, as antioxidants.

Advantageously according to the invention at least one compound corresponding to formula I is used

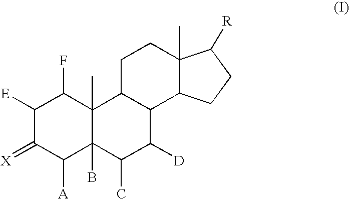

in which

X represents an oxime group (═NOH);

R represents a group chosen from

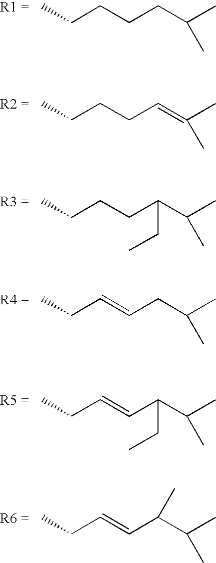

A represents a hydrogen atom or together with B a carbon-carbon bond

B represents a hydrogen atom, a hydroxy group or together with A a carbon-carbon bond,

C represents a hydrogen atom, a ketone group or an oxime group (═NOH), or together with D a carbon-carbon bond,

D represents a hydrogen atom or together with C a carbon-carbon bond,

E represents a hydrogen atom or together with F a carbon-carbon bond,

F represents a hydrogen atom or together with E a carbon-carbon bond, or one of its addition salts with acceptable acids, or one of its esters or one of the addition salts with acceptable acids of said esters, as an antioxidant.

The compounds of formula I as defined above are described in the international application published on 30^{th}Sep. 2004 under number WO 2004/082581 as well as in the French application published on 22/06/2007 under number FR2894968.

Advantageously, according to the invention at least one compound of formula I is used, chosen from the compounds for which, as X represents an oxime group (═NOH) then:
A represents together with B a carbon-carbon bond, C, D, represent a hydrogen atom, E, F represent a hydrogen atom or together a carbon-carbon bond and R has the meaning R1,
A represents together with B a carbon-carbon bond, C, D represent a hydrogen atom, E, F represent a hydrogen atom and R has the meaning R2 or R3 or R4,
A represents together with B a double bond, C represents together with D a carbon-carbon bond, E, F represent a hydrogen atom and R has the meaning R1 or R6,
A represents together with B a double bond, C represents together with D a carbon-carbon bond, E represents together with F a carbon-carbon bond and R has the meaning R1,
E represents together with F a double bond, C, D, A, B represent a hydrogen atom, and R has the meaning R1,

or one of its addition salts with acceptable acids, or one of its esters or one of the addition salts with acceptable acids of said esters.

Still more advantageously according to the invention cholestan-3-one oxime, cholest-4-en-3-one oxime, cholest-1,4-dien-3-one oxime, is used, very preferably cholest-4-en-3-one oxime or cholest-1,4-dien-3-one oxime, or one of its addition salts with pharmaceutically acceptable acids, or one of its esters or one of the addition salts with pharmaceutically acceptable acids of said esters.

According to the invention, the addition salts with pharmaceutically acceptable acids can be for example salts formed with hydrochloric, hydrobromic, nitric, sulphuric, phosphoric, acetic, formic, propionic, benzoic, maleic, fumaric, succinic, tartaric, citric, oxalic, glyoxylic, aspartic or alkane sulphonic acid such as methane or ethane sulphonic, or arylsulphonic acid, such as benzene or paratoluene sulphonic, or carboxylic acids.

It is understood according to the invention that the oxime group represents the two syn and anti isomers in a mixture or isolated.

Of course according to the invention it is possible to use the oxime derivative of cholest-4-en-3-one alone or in a mixture with at least one other oxime derivative of cholest-4-en-3-one.

It is also possible to use the oxime derivatives of cholest-4-en-3-one alone or in a mixture as described previously in combination with one or more other compounds known for their antioxidant properties.

There may be mentioned as examples of other compounds known for their antioxidant properties, the compounds originating from the families of the thiols and the phenols and polyphenols such as for example flavonoids (very widespread in vegetables), phenolic acids (in cereals, fruits and vegetables), tannins (in cocoa, coffee, tea, grapes, etc.), anthocyans (in particular in red fruits; β-carotene (provitamins A); the tocopherols (vitamin E) or its esters such as alpha-tocopherol, gamma-tocopherol, delta-tocopherol; certain metal chelating agents or ascorbic acid and its esters such as sodium or calcium ascorbate; diacetyl 5-6-1-ascorbic acid, palmityl 6-1-ascorbic acid, citric acid and citrates such as sodium, potassium and calcium citrates; tartaric acid and tartrates such as sodium and potassium tartrates; butylhydroxyanisol and butylhydroxytoluol; octyl or dodecyl gallates; sodium, potassium or calcium lactates; lecithins; glutathione, or enzymes such as catalase, the superoxide dismutases and certain peroxidases.

The antioxidants which can be used in the composition of the invention can be natural or synthetic.

Thus, one of the aspects of the invention is therefore to propose an antioxidant cosmetic composition comprising in a cosmetically acceptable medium at least an effective quantity of at least one oxime derivative of cholest-4-en-3-one.

A subject of the invention is also a cosmetic composition intended to combat chronobiological and/or light-induced ageing comprising, in a cosmetically acceptable medium, an effective quantity of at least one oxime derivative of cholest-4-en-3-one.

By cosmetically acceptable medium is meant compatible with the skin, scalp, mucous membranes, nails and hair.

The quantity of oxime derivative of cholest-4-en-3-one or of one of its derivatives which can be used according to the invention obviously depends on the sought effect and must be in an effective quantity in order to produce the sought antioxidant effect.

By way of example the quantity of at least one oxime derivative of cholest-4-en-3-one or of its derivatives which can be used according to the invention can range for example from 0.01% to 30% and preferably from 0.1% to 10% of the total weight of the composition.

The composition according to the invention obviously comprises a cosmetically acceptable support and can be presented in all the galenic forms normally used, particularly for a topical application. Thus the composition can be presented in particular in the form of an aqueous, hydroalcoholic or oily solution, an oil-in-water or water-in-oil or multiple emulsion, an aqueous or oily gel, an anhydrous liquid, pasty or solid product, a dispersion of oil in an aqueous phase using spherical particles, these spherical particles being able to be polymeric nanoparticles such as nanospheres and nanocapsules or, better, ionic and/or non-ionic type lipid vesicles.

This composition can be more or less fluid and have the appearance of a white or coloured cream, an ointment, milk, lotion, serum, paste or foam. It can optionally be applied to the skin in the form of an aerosol. It can also be presented in solid form, and for example in stick form.

The composition of the invention can be used as a care product, as a cleansing product, as a make-up product or also as a simple deodorant product.

In a known manner, the composition of the invention can also contain the usual adjuvants in the cosmetics and dermatological fields, such as hydrophilic or lipophilic gelling agents, hydrophilic or lipophilic active ingredients, preservatives, solvents, fragrances, fillers, filters, pigments, chelating agents, odour absorbers and colorants. The quantities of these different adjuvants are those used in a standard fashion in the fields considered, and for example from 0.01% to 20% of the total weight of the composition. These adjuvants, according to their nature, can be introduced into the oil phase, the aqueous phase, lipid vesicles and/or nanoparticles.

When the composition of the invention is an emulsion, the proportion of the oil phase can range from 5% to 80% by weight, and preferably from 5% to 50% of the total weight of the composition. The oils, the emulsifying agents and the coemulsifying agents used in the composition in the form of emulsion are chosen from those used in a standard fashion in the field considered. The emulsifying agent and the coemulsifying agent are present, in the composition, in a proportion ranging from 0.3% to 30% by weight, and preferably from 0.5% to 20% of the total weight of the composition.

As oils which can be used in the invention, there may be mentioned mineral oils, oils of vegetable origin (apricot oil, sunflower oil, shea butter), oils of animal origin, synthetic oils, silicone oils and fluorinated oils (perfluoropolyethers). It is also possible to use as fats, fatty alcohols (cetyl alcohol), fatty acids, waxes (beeswax).

As emulsifying agents and coemulsifying agents which can be used in the invention, there may be mentioned for example fatty acid and polyethylene glycol esters such as PEG-40 stearate, PEG-100 stearate, fatty acid and polyol esters such as glyceryl stearate and sorbitan tristearate.

As hydrophilic gelling agents, there may be mentioned in particular the carboxyvinyl polymers (carbomers), acrylic copolymers such as the acrylate/alkylacrylate copolymers, the polyacrylamides, the polysaccharides, natural gums and clays, and, such as lipophilic gelling agents, there may be mentioned modified clays such as the bentones, metal salts of fatty acids, hydrophobic silica and the polyethylenes.

The composition can contain other hydrophilic active ingredients such as proteins or protein hydrolysates, amino acids, polyols, urea, allantoin, sugars and sugar derivatives, water-soluble vitamins, vegetable extracts and hydroxy acids.

As lipophilic active ingredients, it is possible to use retinol (vitamin A) and derivatives thereof, tocopherol (vitamin E) and derivatives thereof, essential fatty acids, ceramides, essential oils, salicylic acid and derivatives thereof.

It is also possible to use, according to the invention, in combination with at least one oxime derivative of cholest-4-en-3-one or one of its derivatives, compounds chosen from:
vegetable hormones (auxins);
antibacterial agents such as the macrolides, pyranosides and tetracyclines, and in particular erythromycin;
calcium antagonists, such as verapamil and diltiazem;
OH radical scavengers, such as dimethyl sulphoxide;
vegetable extracts such as those of Iridaceae or soya, extracts which may or may not contain isoflavones;
extracts of micro-organisms including in particular bacterial extracts such as those of non-photosynthetic filamentous bacteria.

Other compounds can also be added to the above list, namely for example potassium channel openers such as diazoxide and minoxidil, spiroxazone, phospholipids such as lecithin, linoleic and linolenic acids, salicylic acid and derivatives thereof described in the French patent FR 2 581 542, such as the salicylic acid derivatives bearing an alkanoyl group having 2 to 12 carbon atoms in position 5 of the benzene ring, hydroxycarboxylic or cetocarboxylic acids and esters thereof, lactones and their corresponding salts, anthralin, carotenoids, the eicosatetraenoic and eicosatrienoic acids or esters and amides thereof, vitamin D and derivatives thereof.

According to the invention, it is possible, inter alia, to combine the oxime derivative of cholest-4-en-3-one with other active ingredients intended in particular for the prevention and/or treatment of cutaneous diseases. Among these active ingredients, there may be mentioned by way of example:
agents modifying cutaneous differentiation and/or proliferation and/or pigmentation such as retinoic acid and its isomers, retinol and its esters, vitamin D and derivatives thereof, oestrogens such as oestradiol, kojic acid or hydroquinone;
agents modifying bacterial adhesion to the skin and/or mucous membranes such as honey, in particular acacia honey and certain sugar derivatives;
antiparasitics, in particular metronidazole, crotamiton or pyrethrinoids;
antifungals, especially compounds belonging to the imidazole class, such as econazole, ketoconazole or miconazole or salts thereof, polyene compounds such as amphotericin B, compounds of the allylamine family such as terbinafine, or also octopirox;
antiviral agents such as acyclovir;
steroidal anti-inflammatory agents such as hydrocortisone, betamethasone valerate or clobetasol propionate, or nonsteroidal anti-inflammatory agents such as ibuprofen and salts thereof, diclofenac and salts thereof, acetylsalicylic acid, paracetamol or glycyrrhetinic acid;
anaesthetic agents such as lidocaine hydrochloride and derivatives thereof;
antipruritic agents such as thenaldine, trimeprazine or cyproheptadine;
keratolytic agents such as alpha- and beta-hydroxycarboxylic or beta-ketocarboxylic acids, their salts, amides or esters, and more particularly hydroxy acids such as glycolic acid, lactic acid, malic acid, salicylic acid, citric acid and the fruit acids in general, and 5-n-octanoylsalicylic acid;
antiseborrhoeics such as progesterone;
antidandruff agents such as octopirox or zinc pyrithione;
anti-acne agents such as retinoic acid or benzoyl peroxide.
substances such as substance P antagonists, CGRP antagonists or bradykinin antagonists or NO synthase inhibitors, compounds described as being active in the treatment of sensitive skins and as having anti-irritant effects, in particular vis-à-vis irritant compounds which may be present in the compositions.

Thus, another subject of the invention relates to a composition comprising an effective quantity of at least one cholest-4-èn-3-one oxime and at least one agent chosen from the antibacterial, antiparasitic, antifungal, antiviral, anti-inflammatory, antipruritic, anaesthetic, keratolytic, antiseborrhoeic, antidandruff, anti-acne agents, agents modifying cutaneous differentiation and/or proliferation and/or pigmentation, substance P antagonists, CGRP antagonists or bradykinin antagonists or NO synthase inhibitors.

As active ingredients, it is possible to use in particular moisturizers such as polyols (for example glycerine), vitamins (for example D-panthenol), anti-inflammatory agents, soothing agents (allantoin, cornflower water), UVA and UVB filters, mattifying agents (for example the partially crosslinked polydimethylorganosiloxanes sold under the name KSG® by Shin Etsu), and mixtures thereof.

Antiwrinkle active ingredients can also be added, in particular tensor products such as vegetable proteins and their hydrolysates, in particular the soya protein extract sold under the name Eleseryl® by LSN or the oat derivative sold under the name Reductine® by Silab.

Other characteristics and advantages of the invention will become clearer from the following examples, given by way of a non-limitative illustration. In what follows, or in the above, the proportions are given in percentage by weight, unless otherwise indicated.

The following examples illustrate the present application without however limiting it.

### EXAMPLE 1

### Competition of cholest-4-en-3-one oxime-3-ol with 5,5-dimethyl-1-pyrroline-1-oxide in the Presence of Free Radicals

The antiradical properties of the claimed products are demonstrated by carrying out a competition study with a reference product belonging to the nitrone family. The nitrones such as DMPO (5,5-dimethyl-1-pyrroline-1-oxide) are broadly described as being compounds exhibiting very high reactivity vis-à-vis the free radicals (Novelli G. P. et al. Free Radical Res. Commun. 1986, 1, 321). The nitrones trap the radical species (R.; RO⁻) and allow their observation by electron paramagnetic resonance (EPR) (Degray J. et al. Electron Spin Resonance, Ed N.M. Atherton, Atheaeum Press Ltd; Cambridge, 1994, 14, 246).

The incubation of the DMPO (Interchim-U2469) (20 mM) in deoxygenated toluene (Sigma-Aldrich) in the presence of the tBuO (tert-butoxyl) radical, generated by photolysis, makes it possible to identify and quantify by EPR the signal of the DMPO-tBuO radical. This signal is inhibited in the presence of an equimolar quantity of cholest-4-en-3-one oxime.

The EPR signal allows integration in the form of an area of the signal of the DMPO-tBuO adduct and therefore a relative quantification of this radical entity.

All the experiments were carried out on an X-band Bruker ESP300 device (9.5 GHz) at ambient temperature. The solutions were studied in a quartz EPR tube.

The data are presented as figures in the table below.

| | Area of the DMPO- | Area of the DMPO- |
|---|---|---|
| t-BuO.: (t-BuO)₂ 20 mM | tBuO signal at 200 s | tBuO signal at 400 s |
| photolysis at 350 nm | in relative units | in relative units |
| DMPO (20 mM) alone | 6.0 | 9 |
| DMPO (20 mM) + cholest- | 3 | 5.5 |
| 4-en-3-one oxime (20 mM) | | |
| % inhibition of the DMPO- | 50% | 40% |

tBuO radical

### CONCLUSION

The cholest-4-en-3-one oxime inhibits of the order of 50% of the level of the DMPO-tBuO signal radical with rapid kinetics, less than 10 minutes. The intensity of the scavenging of the tBuO radical and its kinetics demonstrate the anti-radical and therefore antioxidant property of the claimed compounds.

### EXAMPLE 2

### Antioxidant Effect of cholest-4-èn-3-one oxime in the Model of Oxidation of Cumene by Activated Oxygen

In order to demonstrate the relevance of the antioxidant effect of cholest-4-èn-3-one oxime, the inhibition of the oxidation of the cumene in hydroperoxycumene was studied. This test shows the benefit of involving the biologically most relevant oxidant, i.e. gaseous oxygen. The oxidation of cumene at atmospheric pressure and at 37° C. by oxygen is known and described in the presence of a radical initiator such as AIBN (azobisisobutyronitrile) (Blanchard H. S., J. Am. Chem. Soc. 1959, 81, 4548). A recent publication used this reaction to classify the antioxidant potential of known products such as vitamin E (which is the universal reference), BHT (butylated hydroxytoluene) and other products.

In the following experiment we reproduced the same experimental conditions as those described in the publication of Becker D. A. et al. (J. Am. Chem. Soc. 2002, 124, 4678-4684). A high pressure liquid chromatography method coupled with a UV detector was used to detect cumene hydroperoxide. The column used is an Agilent Zorbax Eclipse XDB RPC8 column (150×4.6 mm) coupled with a UV detector fixed at 254 nm. The gradient used is detailed in the following table:

| Time (min) | % water | % Acetonitrile | Flow rate ml/min |
|---|---|---|---|
| 0 | 35 | 65 | 1.5 |
| 5 | 35 | 65 | 1.5 |
| 5.5 | 0 | 100 | 1.5 |
| 10 | 0 | 100 | 1.5 |

Experimental Conditions:

2 ml of cumene (AcrosOrganic) and 0.5 ml of methanol are mixed, azobisisobutyronitrile (AcrosOrganic) is added (2 equivalents) and the solution taken to 45° C. in order to accelerate the chemical reaction. The cumene hydroperoxide appears over time and is assayed by the HPLC method.

A straight line of linear regression is established in order to demonstrate that the quantification of the appearance of cumene hydroperoxide is possible. The results are shown in the following table:

| Cumene hydroperoxide concentration | |
|---|---|
| (mM) | Time (min) |
| 0.25 | 0 |
| 1 | 15 |
| 1.5 | 30 |
| 2.2 | 45 |
| 3 | 60 |
| 3.7 | 75 |

The straight line of linear regression obtained corresponds to the following formula: y=0.0444x+0.2694 with R²=0.994

The technique used made it possible to compare the appearance of the cumene hydroperoxide in the presence of an antioxidant agent such as the claimed products and vitamin E used as reference product.

The experimental data and the results in percentages of the oxidation of the cumene are presented in the table below.

| | | Area of the | |
|---|---|---|---|
| | | signal, in | % of reduction |
| | | relative units, | of the |
| Experimental | | of the cumene | oxidation of |
| conditions: | Incubation | hydroperoxide | the cumene |
| Cumene − Methanol + | 2 hours | 250 | 38% |
| 100 μM of cholest-4-en-3- | 45° C. | | |
| one oxime and 2 | | | |
| equivalents of AIBN | | | |
| Cumene − Methanol + | 2 hours | 175 | 57% |
| 200 μM of cholest-4-en- | 45° C. | | |
| 3-one oxime and 2 | | | |
| equivalents of AIBN | | | |
| Cumene − Methanol + | 2 hours | 245 | 39% |
| 100 μM of vitamin E and | 45° C. | | |
| 2 equivalents of AIBN | | | |
| Cumene − Methanol and | 2 hours | 400 | 0% (positive |
| 2 equivalents of AIBN | 45° C. | | control) |

In conclusion cholest-4-èn-3-one oxime reduces by approximately 40% the oxidation of the cumene by oxygen and exhibits an activity similar to vitamin E under these conditions at the same concentrations.

### EXAMPLE 3

### Toxicology Study Carried Out with cholest-4-èn-3-one oxime

A general toxicity study was carried out on Beagles receiving cholest-4-èn-3-one oxime in suspension in corn oil by oral administration once daily for 39 weeks. The detailed toxicological analysis of this study made it possible to describe a dose with a no-observed-effect level (NOEL) at 50 mg/kg. This result demonstrates this product's very high safety.

### EXAMPLE 4

### Study of Pharmacokinetics in the Dog with cholest-4-èn-3-one oxime

A study of pharmacokinetics with administration by oral route in suspension in corn oil and by intra-venous route in solution in cremophor/ethanol/water (5%, 10%, 85%) made it possible to calculate the bioavailability of the product. The assay of the product in the plasma is carried out by a high pressure liquid chromatography method coupled with mass spectrometry.

Thus, at a dose of 500 mg/kg administered by oral route, the bioavailability was calculated at 6%. The circulating level of cholest-4-en-3-one oxime is therefore quantifiable and demonstrates a real absorption of the product.

### EXAMPLE 5

### Chemical Stability of cholest-4-en-3-one oxime

A study of chemical stability under the storage conditions described in the ICH standards (International Conference on Harmonization of Technical Requirements for Registration of Pharmaceuticals for Human Use) for the product stored in the state of powder demonstrated very high chemical stability. These analyses were carried out using a high pressure liquid chromatography method coupled with a UV detector. This method makes it possible to quantify the impurities at a level of 0.05%. The results (total impurities as a function of time) are shown in the following table.

| Storage conditions | T0 | T6 months | T24 months |
|---|---|---|---|
| 25° C. under 60% relative humidity | 1.8% | — | 1.8% |
| 40° C. under 75″% relative humidity | 2.1% | 1.9% | — |

No change in the quality of the product appeared after 6 months of storage at 40° C. and above all after 2 year's storage at 25° C.

1. A method for preventing and/or treating damage caused by oxidative free radicals, said method comprising administering to a subject in need thereof an effective amount of at least one antioxidant compound chosen from: an oxime derivative of cholest-4-en-3-one, or an addition salt thereof with acceptable acids, or an ester thereof, or an addition salt of said ester with acceptable acids.

2. The method of claim 1, wherein said at least one antioxidant compound comprises a compound of the following formula I: 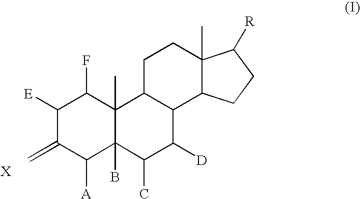
in which
X represents an oxime group (═NOH);
R represents a group chosen from 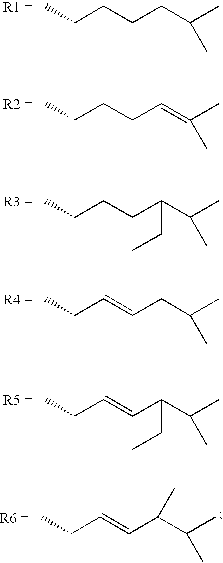
A represents a hydrogen atom, a hydroxy group or together with B a carbon-carbon bond;
B represents a hydrogen atom, a hydroxy group or together with A a carbon-carbon bond;
C represents a hydrogen atom, a ketone group or an oxime group (═N—OH), or together with D a carbon-carbon bond;
D represents a hydrogen atom or together with C a carbon-carbon bond;
E represents a hydrogen atom or together with F a carbon-carbon bond;
F represents a hydrogen atom or together with E a carbon-carbon bond;
or an addition salt thereof with pharmaceutically acceptable acids, or an ester thereof or an addition salt thereof with pharmaceutically acceptable acids of said esters.

3. The method of claim 2, wherein the compound of formula (I) is chosen from compounds for which, as X represents an oxime group (═NOH), then:
A represents together with B a carbon-carbon bond, C, D represent a hydrogen atom, E, F represent a hydrogen atom or together a carbon-carbon bond and R has the meaning R1;
A represents together with B a carbon-carbon bond, C, D represent a hydrogen atom, E, F represent a hydrogen atom and R has the meaning R2 or R3 or R4;
A represents together with B a double bond, C represents together with D a carbon-carbon bond, E, F represent a hydrogen atom and R has the meaning R1 or R6;
A represents together with B a double bond, C represents together with D a carbon-carbon bond, E represents together with F, a carbon-carbon bond and R has the meaning R1; or
E represents together with F a double bond, C, D, A, B represent a hydrogen atom, and R has the meaning R1.

4. The method of claim 2, wherein the compound of formula (I) is chosen from cholestan-3-one oxime, cholest-4-en-3-one oxime, or cholest-1,4-dien-3-one oxime.

5. The method of claim 1, wherein the compound of formula (I) is chosen from cholest-4-en-3-one oxime or cholest-1,4-dien-3-one oxime.

6. The method of claim 1, wherein the at least one antioxidant compound is administered in a cosmetic.

7. The method of claim 6, to combat oxidative stress.

8. The method of claim 6, to treat ageing, and/or cutaneous ageing.

9. The method of claim 8, for treating fine lines and deep wrinkles, modifications of the skin tone, the dry and rough appearance of the skin, the loss of firmness and/or tonicity of the skin.

10. The method of claim 1, wherein the at least one antioxidant compound is administered in a food.

11. The method of claim 10, wherein the at least one antioxidant compound is a food supplement.

12. The method of claim 1, wherein the at least one antioxidant compound is an antioxidant preservative in a cosmetic, food or pharmaceutical product.

13. The method of claim 1, wherein the oxime derivatives of cholest-4-en-3-one are used alone or in a mixture, optionally in combination with one or more other antioxidant compounds.

14. A method for preserving a cosmetic, pharmaceutical, or food product, said method comprising contacting said product an effective amount of at least one antioxidant compound chosen from: an oxime derivative of cholest-4-en-3-one, or an addition salt thereof with acceptable acids, or an ester thereof, or an addition salt of said ester with acceptable acids.
